(19) United States
(12) Patent Application Publication
(10) Pub. No.: US 2010/0331723 A1
(43) Pub. Date: Dec. 30, 2010

(54) Two-Point Discriminator

(75) Inventors: N.R. Chandrasekar, Canton, MA (US); Donald McKay, Belmont, MA (US)

Correspondence Address: GOODWIN PROCTER LLP;PATENT ADMINISTRATOR, 53 STATE STREET, EXCHANGE PLACE, BOSTON, MA, 02109-2881, US

(73) Assignee: IQ Medical Devices, Belmont, MA (US)

(21) Appl. No.: 12/865,214

(22) PCT Filed: Jan. 30, 2009
(86) PCT No.: PCT/US09/32548

§ 371 Date: Jul. 29, 2010

Related U.S. Application Data
(60) Provisional application No. 61/024,680, filed on Jan. 30, 2008.

(51) Int. Cl. A61B 19/00

(52) U.S. Cl. 600/557

(57) ABSTRACT

The invention relates to a design for an instrument for measuring two-point discrimination between two blunt points. The instrument includes a housing, a first needle, a second needle, and a mechanism for providing movement to at least one of the needles.

### CROSS-REFERENCE TO RELATED APPLICATIONS

This application claims priority to and the benefit of U.S. Provisional Application Ser. No. 61/024,680, filed Jan. 30, 2008, the disclosure of which is being incorporated herein by reference in its entirety.

### FIELD OF THE INVENTION

This invention relates generally to an instrument for measuring sensation in a digit or appendage, and more particularly to an instrument for measuring sensory discrimination between two points on the digit.

### BACKGROUND

The measurement of sensation in the fingers and toes is an important part of the physical examination of patients in which impairment of sensation is suspected and needs to be objectively confirmed. This impairment can be secondary to multiple factors ranging from intrinsic neurologic disease (e.g., diabetic neuropathy) to nerve entrapments (e.g., carpal tunnel syndrome) to traumatic injuries of the nerves themselves, such as lacerations or crush injuries.

An accurate measurement of two-point discrimination will serve to confirm the diagnosis of nerve injury. It can also measure recovery of the nerve from an injury. The testing is carried out on the pads of the fingers and/or the toes where the majority of the sensory receptors are located. The measurement is based on the ability of the normally innervated pad to distinguish between two blunt points that are 4 to 6 mm apart. An equivocal result would be between 6 mm and 10 mm. A definitely abnormal result would be an inability to distinguish between two points that are more than 10 mm apart. The results of such an examination, if performed accurately using proper instrumentation, will be important in helping to make a diagnosis. This test can also help in evaluating recovery from a nerve injury.

Although there are several instruments used by doctors to measure two-point discrimination, some are awkward to carry, hard to use, and/or easily misplaced. In many cases, the examiner uses a bent paper clip, which may be inaccurate. This has obvious deficiencies unless the points of the paper clip are carefully calibrated. An instrument has been developed called the Disk-Criminator® which provides accurate sensory measurements. It is, however, somewhat awkward to carry, because of its circular shape. Another available instrument is a variation of a standard digital caliper in which the points are moved along a marked sidebar; however, this instrument is also difficult to carry and awkward to use.

### SUMMARY

The present invention is generally directed to an instrument for measuring two-point discrimination between two blunt points which can be placed at anywhere from about 2 mm (0.08 inches) to about 12 mm (0.5 inches) apart. The testing is typically carried out on the pads of the fingers and the toes, as this is the area where the majority of the sensory receptors are located; however, the instrument can be used to measure two-point discrimination on other regions of the body. The instrument would typically be used by hand surgeons, emergency room physicians and medical students. The instrument could also be used by occupational therapists, neurologists and residents and fellows in neurology, hand surgery, plastic surgery and orthopedic surgery.

Generally, the instrument is sized and shaped to be easily held and manipulated with one hand while being used. The instrument is capable of performing accurate measurements of sensory discrimination. In one embodiment, the instrument is about 12.5 cm to about 17.5 cm (5-7 inches) in length, about 12 mm to about 20 mm (0.5 to 0.75 inches) in width, and about 6 mm to about 12 mm (0.25 to 0.5 inches) in depth. On transverse cross-section, the instrument has a generally rectangular in shape; however, other shapes are contemplated and within the scope of the invention, such as, for example, square, round, or elliptical. On one end of the instrument, for example the end where the two blunt points are, there may be a gauge for indicating the distance between the two points. The instrument also includes a mechanism that controls the distance between the two points. The mechanism may be a rotary or linear actuator that can be moved by one's thumb or finger. The mechanism may include easily visible markings for tracking the movement and relative distance of the two points.

Additionally, the instrument may include such optional features as a pocket clip, a light, a scale or ruler on one side of the instrument, and a protective cover. The pocket clip facilitates carrying the instrument in the pocket of a shirt, suit or a lab coat. The protective cover for the points may be retractable and/or removable.

In one aspect, the invention relates to a medical instrument for evaluation of two-point discrimination. The instrument includes an elongate body member or housing, a first needle having a distal tip, the first needle fixedly coupled to and extending from a distal portion of the elongate body member, a second needle having a distal tip, and a mechanism coupled to the elongate body member and the second needle. The second needle is movably coupled to the elongate body member via the mechanism. The mechanism is configured to move the distal tip of the second needle relative to the distal tip of the first needle.

In various embodiments of the foregoing aspect, the mechanism can be disposed at least partially within the elongate body member. The instrument can include a gauge configured to indicate a distance between the distal tip of the first needle and the distal tip of the second needle. In one embodiment, the gauge is operatively coupled to the mechanism. The range of movement of the distal tip of the second needle relative to the distal tip of the first needle is from about 0.5 mm (0.02 inches) to about 20 mm (0.8 inches), preferably about 1 mm (0.04 inches) to about 14 mm (0.6 inches), and more preferably about 2 mm (0.08 inches) to about 12 mm (0.5 inches). In addition, the instrument can include a cover slidably coupled to the elongate body member, where the cover is slidable between a first position at least partially covering the first and second needles and a second position exposing the first and second needles. In further embodiments of the instrument, the mechanism includes at least one of a worm drive, a rotary actuator, a linear actuator, a gear train, a linkage, a cam and follower, a bifurcated beam and splitter arrangement, or combinations thereof. The movement of the second needle relative to the first needle can be by at least one of linear displacement and angular displacement. In addition, the needles can have a shape, such as linear, arcuate (e.g., having at least one curve), or combinations thereof. In one embodiment, the first and second needles have an arcuate shape.

In addition, the instrument can include an illumination source. The illumination source can be, for example, a light emitting diode. In one embodiment, the illumination source is disposed on a proximal portion of the elongate body member. The instrument can also include, for example, a pocket clip, a scale disposed along a side of the elongate body member, and a power source. The instrument can also include a goniometer and light emitting diode (LED) indicators. In one embodiment, the power source is a battery. The elongate body member can be manufactured from a molded plastic, suitable metals, or combinations thereof. The elongate body member can be manufactured as a single housing or as multiple parts that are assembled, for example, snap-fit together. In addition, the elongate body member can be a substantially solid body defining recesses formed therein to accommodate the various components, for example, the mechanism and the battery.

In another aspect, the invention relates to a medical instrument for evaluation of two-point discrimination. The instrument includes an elongate body member, a mechanism coupled to the elongate body member and at least partially disposed in a distal portion thereof, a first needle coupled to the mechanism and extending from the distal portion of the elongate body member, and a second needle coupled to the mechanism and extending from the distal portion of the elongate body member. The mechanism is configured to provide movement to at least one of the first needle and the second needle to vary a distance between the first needle and the second needle.

In yet another aspect, the invention relates to a medical instrument for evaluation of two-point discrimination. The instrument includes an elongate body member, a mechanism coupled to and at least partially disposed in the elongate body member, a first needle having a distal tip, the first needle coupled to the mechanism and extending from a distal portion of the elongate body member, a second needle having a distal tip, the second needle coupled to the mechanism and extending from the distal portion of the elongate body member, and a gauge disposed on the elongate body member. The gauge can be configured to indicate a distance between the distal tip of the first needle and the distal tip of the second needle. The mechanism is configured to provide movement to at least one of the first needle and the second needle to vary a distance between the first needle and the second needle.

In various embodiments of the foregoing aspects of the invention, the mechanism is configured to provide substantially symmetrical movement of the first needle and the second needle relative to the elongate body member. The mechanism can also be configured to provide substantially concomitant movement of the first needle and the second needle. The mechanism can be at least one of a worm drive, a rotary actuator, a linear actuator, a gear train, a linkage, a cam and follower, a bifurcated beam and splitter arrangement, or combinations thereof.

These and other objects, along with the advantages and features of the present invention herein disclosed, will become apparent through reference to the following description, the accompanying drawings, and the claims. Furthermore, it is to be understood that the features of the various embodiments described herein are not mutually exclusive and can exist in various combinations and permutations.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIG. 1 is a schematic plan view of a two-point discriminator instrument in accordance with one embodiment of the invention;

FIG. 2 is a schematic side view of the instrument of FIG. 1;

FIG. 3A is an enlarged schematic plan view in partial cross-section of a portion of the instrument of FIG. 1;

FIG. 3B is an alternative arrangement of the instrument of FIG. 3A;

FIG. 4 is a schematic plan view of a two-point discriminator instrument in accordance with an alternative embodiment of the invention;

FIG. 5 is a schematic side view of the instrument of FIG. 4;

FIG. 6 is an enlarged schematic plan view of an alternative embodiment of the instrument of FIG. 4;

FIGS. 7A-7C are enlarged schematic side, top, and end views of one embodiment of a mechanism for use in the instrument of FIG. 6;

FIG. 8A is a schematic plan view of a two-point discriminator instrument in accordance with another alternative embodiment of the invention;

FIG. 8B is a schematic plan view of an alternative embodiment of the instrument of FIG. 8A;

FIG. 9 is a schematic side view of the instrument of FIG. 8A;

FIG. 10A is a schematic plan view of a two-point discriminator instrument in accordance with another alternative embodiment of the invention;

FIG. 10B is a schematic plan view of an alternative embodiment of the instrument of FIG. 10A;

FIG. 11 is a schematic side view of the instrument of FIG. 10A;

FIG. 12 is an enlarged schematic plan view in partial cross-section of a portion of the instrument of FIG. 10A;

FIGS. 13A and 13B are schematic plan and side views, respectively, of one component of a mechanism for use in the instrument of FIG. 10A, in accordance with one embodiment of the invention;

FIG. 14 is a schematic plan view of a two-point discriminator instrument in accordance with another alternative embodiment of the invention;

FIG. 15A is a schematic plan view of the instrument of FIG. 14 in a first state;

FIG. 15B is a schematic end view of the instrument of FIG. 15A;

FIG. 16A is a schematic plan view of the instrument of FIG. 14 in a second state;

FIG. 16B is a schematic end view of the instrument of FIG. 16A;

FIGS. 17A and 17B are schematic plan and end views, respectively, of one component of a mechanism for use in the instrument of FIG. 14, in accordance with one embodiment of the invention;

FIG. 18 is a schematic exploded end view of the instrument of FIG. 14;

FIG. 19 is a schematic plan view in partial cross-section of an alternative version of the instrument of FIG. 14;

FIG. 20 is a schematic side view of another alternative version of the instrument of FIG. 14;

FIGS. 21A-21C are schematic side, top, and end views respectively of a two-point discriminator instrument in accordance with another alternative embodiment of the invention;

FIG. 22 is an enlarged schematic view of a portion of the top view of FIG. 21B;

FIG. 23 is an enlarged schematic view of the end view of FIG. 21C;

FIG. 24A is an alternative enlarged schematic view of the end view of FIG. 21C; and

FIG. 24B is an enlarged schematic side view of the instrument depicted in FIG. 24A.

### DETAILED DESCRIPTION

In the following, various embodiments of the present invention are described with reference to an instrument to measure sensation in the fingers and toes. It is, however, to be understood that the present invention can also be used to measure two-point discrimination in other appendages.

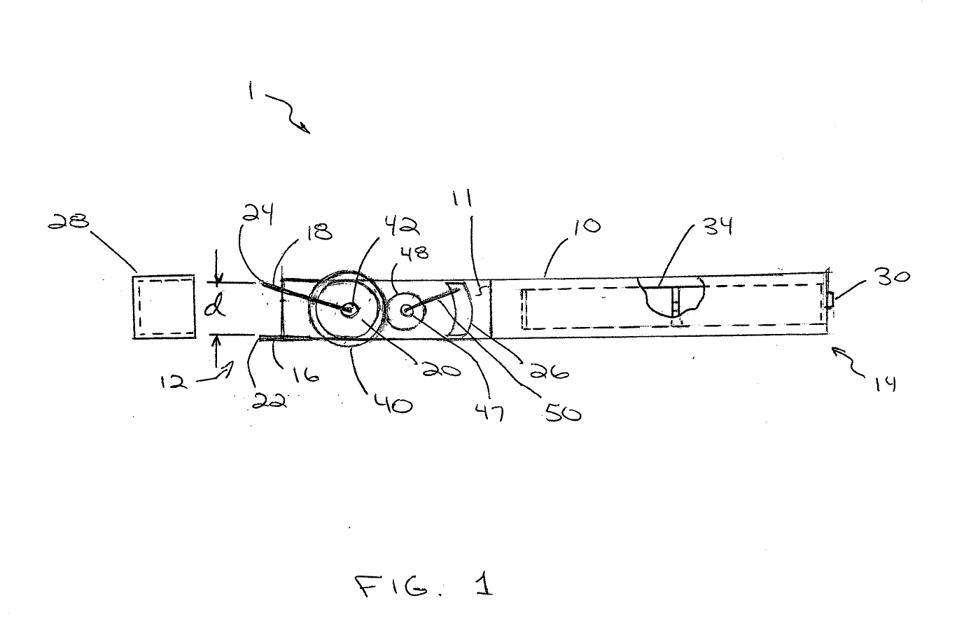

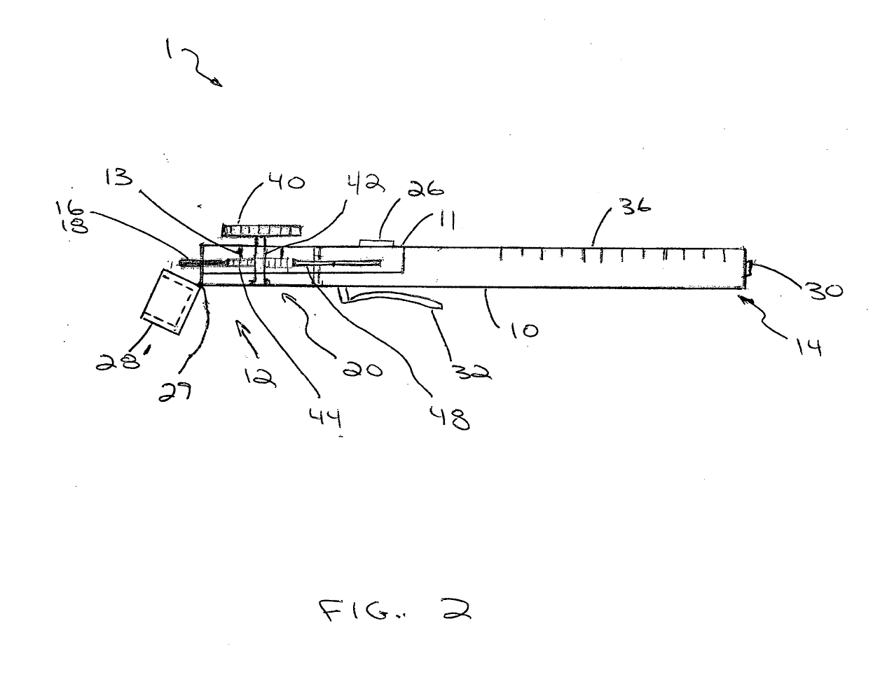

FIGS. 1 and 2 depict one embodiment of a two-point discriminator instrument 1 in accordance with the invention. The instrument 1 includes an elongate body member 10 having a distal end 12 and a proximal end 14. The instrument 1 includes a first needle 16 and a second needle 18 generally located proximate the distal end 12 of the elongate body member 10. The instrument 1 also includes a mechanism 20 disposed on or partially in the elongate body member 10. The mechanism 20 is mechanically coupled to at least one of the first and second needles 16, 18 to provide relative movement thereto.

As shown in FIG. 1, the first needle 16 is fixedly coupled to the distal end 12 of the elongate body member 10, with a distal tip 22 of the first needle 16 projecting beyond the distal end 12 of the elongate body member 10. The first needle 16 acts as a reference point. The first needle 16 can be bonded to or otherwise mechanically coupled to the elongate body member 10. In one embodiment, the needle 16 is press fit into a hole formed in the distal end 12 of the elongate body member 10. Alternatively, the needle 16 can be bonded to an inside wall of the elongate body member 10.

The second needle 18 is coupled to the mechanism 20 and is movable relative to the first needle 16. The second needle 18 also includes a distal tip 24 that extends beyond the distal end 12 of the elongate body member 10. The second needle 18 is coupled to the mechanism 20 so as to be movable relative to the first needle 16 either through angular or linear displacement, as described in greater detail hereinbelow. Displacement (d) is in the range of about 1 mm (0.04 inches) to about 14 mm (0.6 inches), preferably about 2 mm (0.08 inches) to about 12 mm (0.5 inches).

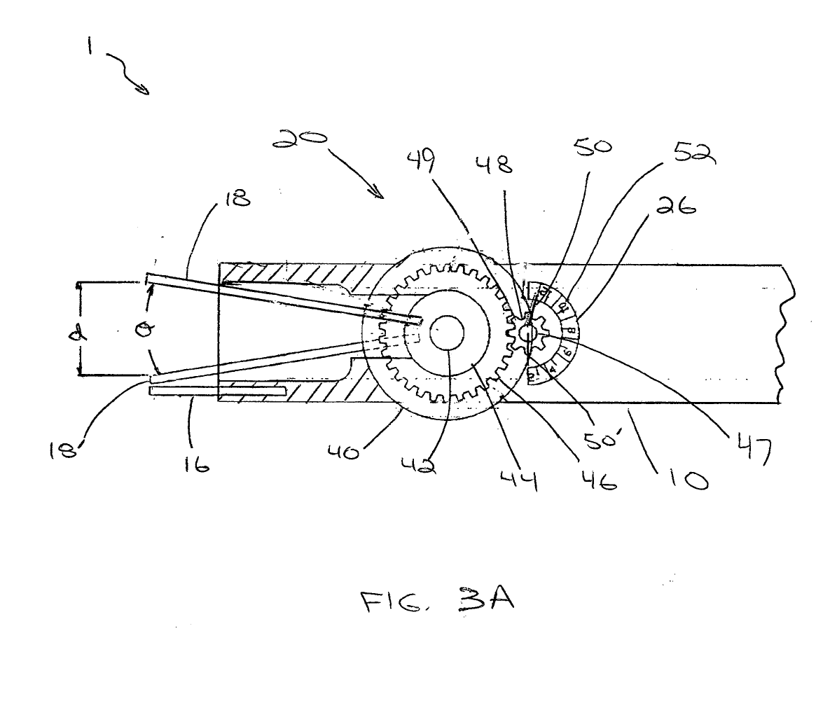

The mechanism 20, which provides movement to the second needle 18, is a gear type mechanism; however, other types of mechanisms are contemplated and within the scope of the invention. The mechanism 20 includes a thumbwheel 40 mounted on the elongate body member 10. The thumbwheel 40 acts as one type of rotary actuator for displacing the second needle 318. The thumbwheel 40 rotates through an arc (\acute{Ø})(see FIGS. 3A and 3B) corresponding to a distance (d), or range of travel, of the second needle 18 relative to the fixed first needle 16, thereby providing angular displacement to the second needle 18. In one embodiment, the thumbwheel 40 is about 1 cm to about 2 cm (0.4-0.8 inches) in diameter and about 4 mm (0.15 inches) in thickness. The thumbwheel 40 can include grooves on its exterior circumference to facilitate turning. In one embodiment, the thumbwheel 40 rotates through an arc of about 27 degrees. The arc corresponding to a distance between the distal tips 22, 24 of the needles 16, 18 of about 12 mm (0.5 inches).

In the center of the thumbwheel 40 is an attached axle 42 connecting the thumbwheel 40 to the elongate body member 10 and a smaller wheel 44. In one embodiment, the axle 42 is about 4 mm (0.16 inches) in diameter and runs through the elongate body member 10. The axle 42 is attached to the sides of the elongate body member 10 by a mounting that allows the axle 42 to rotate freely, for example, a collar and pin arrangement. In one embodiment, the smaller wheel 44 includes teeth on at least a portion of its circumference and is about 1 cm to about 1.5 cm (0.4-0.6 inches) in diameter. The smaller wheel 44 may be a spur gear. Attached to the wheel 44 is the second needle 18 (18′ in the closed position shown in FIG. 3A) also called the sensory pin. The second needle 18 can be attached to the wheel 44 by bonding (e.g., by epoxy or soldering depending on the materials used) or otherwise mechanically coupled (e.g., by a pin or clamp). The size of the needle 18 will vary to suit a particular application and in one example is about 2.5 cm (1.0 inches) in length and about 1 mm (0.04 inches) in diameter with a rounded distal tip 24. In alternative embodiments, the distal tip 24 may be sharp.

In addition, at least one of the thumbwheel 40, axle 42, or wheel 44 are configured to frictionally engage the elongate body member 10 to prevent inadvertent movement of the mechanism 20 when in use. For example, a media insert 13 may be sandwiched between the thumbwheel 40 and a wall of the elongate body member 10. The media insert 13 will cause a frictional resistance to prevent the thumbwheel 40 and second needle 18 from rotating unless the thumbwheel 40 is turned. In one embodiment, the elongate body member 10 is formed of two longitudinal halves that snap together, which facilitates the assembly of the instrument and its internal components. In the embodiment shown in FIG. 2, the elongate body member 10 includes a cover 11 that when removed exposes the components of the mechanism 20. The cover 11 can be snap fit or bonded to the elongate body member 10. When attached, the cover 11 sandwiches the media insert 13 against at least one of the thumbwheel 40, axle 42, or wheel 44 to provide the necessary frictional resistance. The media insert 13 can be sized and shaped to suit a particular application and can be manufactured from a polymer, textile, or metal material.

In the embodiment shown in FIG. 3A, the mechanism 20 includes a gauge 26. The gauge 26 includes a semi-circular scale 52 affixed to or printed on the elongate body member 10 (or cover 11) proximate the thumbwheel 40. In one embodiment, the gauge scale 52 includes markings every 1 mm (0.04 inches) including numbers every 2 mm (0.08 inches) that correspond to the distance between the first and second needles 16, 18. In one embodiment, the scale has a range of about 0 mm to about 12 mm (0-0.5 inches). Optionally, the gauge includes a clear plastic cover over the scale for protection and visualization.

The gauge 26 also includes a spur gear 48 and pin 50 arrangement that mechanically engages the mechanism 20. The gear 48 is attached to the elongate body member 10 by an axle 47, similar to axle 42. The gear 48 is positioned so as the teeth 49 on the gear 48 mesh with the teeth 46 on the wheel 44. The gear 48 in one embodiment is about 4 mm (0.16 inches) in diameter. The gear 48 has a small indicator needle 50 attached to it that interfaces with the gauge scale 52. The indicator needle 50 can be bonded or otherwise mechanically coupled to the gear 48. The arc of the needle 50 rotates on the gauge scale and is the reference scale for the instrument 1 (i.e., indicates the distance between the first and second needles 16, 18). In one embodiment, the needle 50 is about 4 mm (0.16 inches) in length and about 1 mm (0.04 inches) in diameter. The indicator needle 50 rotates through an arc of about 180 degrees. The gear 48 meshes with the teeth 46 on the wheel 44 and is sized such that the approximately 180 degree travel of the indicator needle 50 corresponds to the maximum distance between the first and second needles 16, 18. In one embodiment, the wheel 44 includes a 30T spur gear and the gear 48 is a 8T spur gear.

FIG. 3B depicts an alternative arrangement of the mechanism 20′ with the thumbwheel not shown for clarity. The alternative mechanism 20′ is similar to the mechanism described with respect to FIG. 3A, except for the arrangement of the indicator wheel 48′. In the embodiment shown in FIG. 3B, the indicator wheel 48′ has larger diameter and a greater number of teeth 49′ disposed on the exterior circumference thereof. In this embodiment, the wheel 48′ is about 8 mm (0.3 inches) in diameter with approximately 15 to 24 teeth. The larger wheel 48′ of the embodiment shown in FIG. 3B reduces the angular range of the indicator needle 50 corresponding to the full distance of travel between the first and second needles 16, 18. In this embodiment, the angular range of the indicator needle is less than 90 degrees. As such, only about 50% of the exterior circumference of the wheel 48′ requires teeth 49′ disposed thereon; however, a standard spur gear including teeth along the entire circumference of the wheel 48′ may be used.

The instrument 1 can include other optional components, such as, for example, a cover 28, an illumination source 30, a pocket clip 32, a power source 34, and a scale 36. The optional pocket clip 32 can be located on the side of the elongate body member 10 opposite the location of the mechanism thumbwheel. In the embodiment shown in FIG. 3B, the scale 52′ includes LEDs 37′ disposed proximate the gauge 26′ that correspond to, for example, 2 mm increments along the gauge for further providing indication of the measurement between the first and second needles 16′, 18′. The LEDs can be in electrical communication with a switch or sensor that interacts with one or both of the needles 16′, 18′ and/or one or both of the wheels 44′, 48′. For example, the instrument 1′ can include one or more proximity or limit switches located within the elongate body member 10′ and configured to close and illuminate a corresponding LED when the second needle 18′ contacts or comes into proximity of the switch. The power source previously described can provide the power for the circuit. The exact configuration of the circuit and the arrangement of the switch or sensor will be known to a person of ordinary skill in the art. The LED indicators can be applied to any of the instruments described herein.

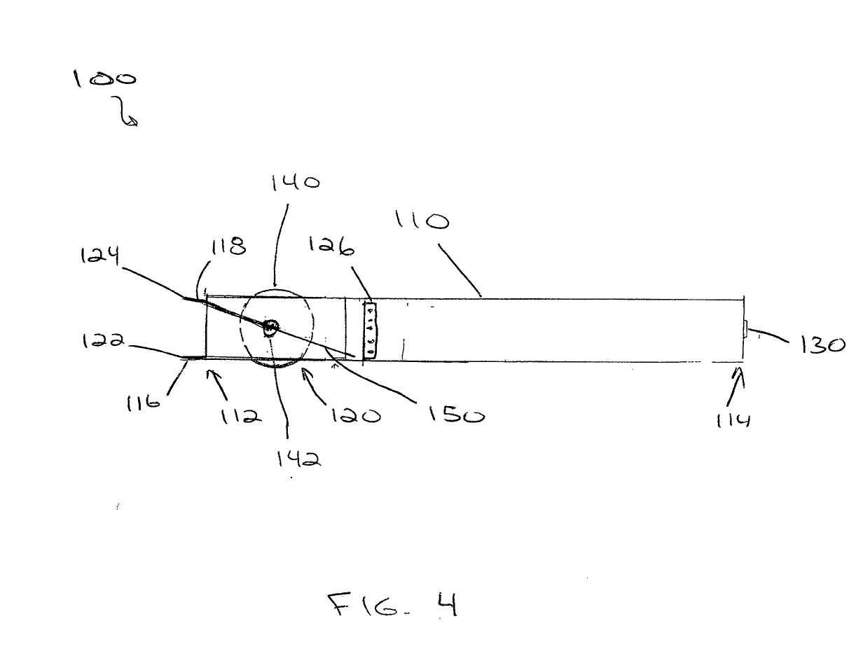

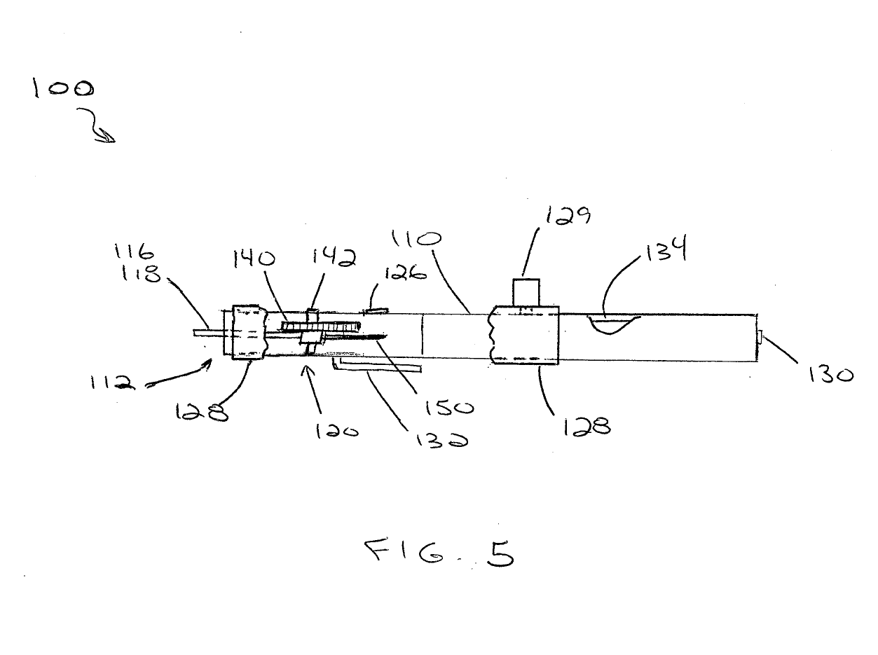

The cover 28 (FIG. 1) can be a retractable plastic protector that slides over the distal end 12 of elongate body member 10 and the distal tips 22, 24 of the needles 16, 18. The cover 28 can be a plastic guard that protects the needles 16, 18 from lateral and up-down stresses and prevents inadvertent exposure to the distal tips 22, 24 of the needles 16, 18, thereby preventing injury. The cover 28 can include a hollow inner cavity to accommodate the needles 16, 18 or could include a slot or transverse groove sized to provide a tighter fit with the needles 16, 18, which would keep the needles 16, 18 in alignment and prevent them from being bent. The cover 28 can include, for example, knurling, notches, or similar structure to facilitate the sliding of the cover 28 on and off of the instrument 1. The cover 28 may include a cable or similar structure to couple the cover 28 to the instrument 1. Alternatively, the cover 28 may be permanently affixed to the elongate body member 10 and be retractable along the elongate body member 10 to expose the distal tips 22, 24 of the needles 16, 18 when retracted (see FIG. 5). The cover 28 can also include notches to accommodate other structures on the elongate body member 10, such as the mechanism 20 or thumbwheel 40. In another alternative embodiment, the cover 28′ (FIG. 2) can be hingedly coupled to the distal end 12 of the elongate body member 10. The hinge 29 can be spring loaded to assist in maintaining the cover in the on or off position relative to the distal end 12 of the instrument 1.

The scale 36 can be molded in, affixed to, or printed on (for example, silk screened) the elongate body member 10 to provide a measuring instrument on the instrument 1. The scale 36 can be disposed on the front face and/or a side surface of the elongate body member 10. The illumination source 30 can be, for example, one or more light-emitting diodes (LED) located on the proximal end 14 of the elongate body member 10. In one embodiment, the LED is a 3 mm white LED; however, other sizes and colors are contemplated and within the scope of the invention. The illumination source 30 can be in electrical communication with the power source 34. In one embodiment, the power source 34 is one or more batteries, for example, two 1.5 volt size AAA alkaline batteries connected in series with the LED.

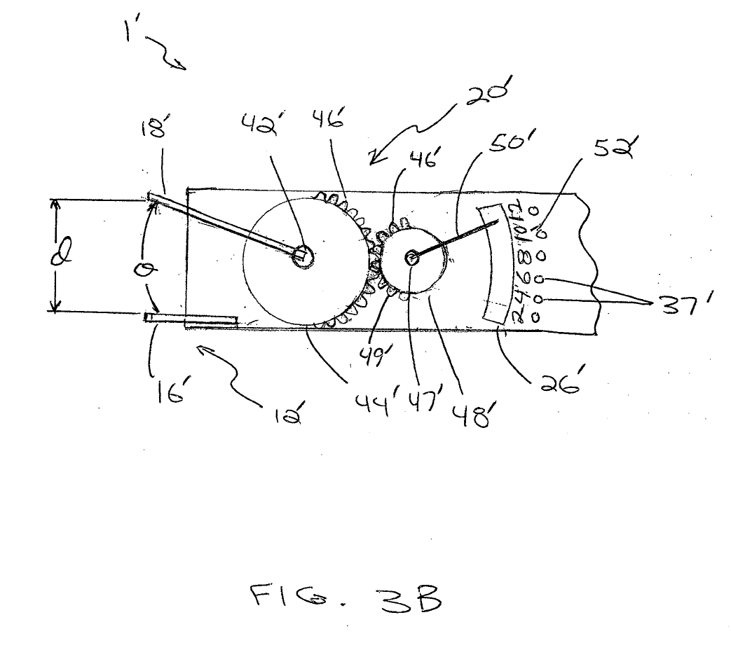

FIGS. 4 and 5 depict an alternative instrument 100 similar to the instrument described hereinabove with respect to FIGS. 1-3. The instrument 100 includes an elongate body member 110, a first needle 116, a second needle 118, a mechanism 120, and a gauge 126. The first needle 116 is fixedly attached to the distal end 112 of the elongate body member 110 and the second needle 118 is coupled to the mechanism 120 and movable relative to the first needle 116.

The mechanism 120 depicted in FIGS. 4 and 5 differs slightly from the mechanism 20 shown in FIGS. 3A and 3B. The mechanism 120 includes a thumbwheel 140 placed inside the elongate body member 110 with a portion of the thumbwheel 140 projecting outside on either side of the elongate body member 110 through, for example, a narrow slit(s) in a side wall(s) of the elongate body member 110. This allows turning of the thumbwheel 140 from either side of the instrument, with, for example, the user's thumb or fingers. In one embodiment, the thumbwheel 140 projects about 3 mm (0.06 inches) beyond the side walls of the elongate body member 110.

The thumbwheel 140 is secured to the elongate body member 110 by an axle 142 passing through the elongate body member 110 and secured to the sides thereof. The thumbwheel 140 and/or axle 142 frictionally engage the elongate body member 110 to prevent inadvertent movement of the thumbwheel 140 and second needle 118. The second needle 118 can be directly mounted on the thumbwheel 140. The second needle 118 can be bonded to or otherwise mechanically coupled to the thumbwheel 140 and/or axle 142. As shown in FIGS. 4 and 5, the second needle 118 can pass through the axle 142 of the wheel and act as the indicator needle 150 on the gauge 126. Alternatively, a separate indicator needle 150 can be attached to the thumbwheel 140 and/or axle 142 opposite the second needle 118. A portion of the distal end 112 of the elongate body member 110 can be transparent to facilitate viewing of the indicator needle 150 relative to the gauge 126. As previously described, the gauge 126 will include markings/numbers indicative of the distance between the distal tips 122, 124 of the first and second needles 116, 118.

The instrument 100 also includes an illumination source 130, a power source 134, a pocket clip 132, and a cover 128, as previously described. In the embodiment shown in FIG. 5, the cover 128 is a retractable type cover 128 that slides towards the distal end 112 of the elongate body member 110 to cover the distal tips 122, 124 of the needles 116, 118 and slides away from the distal end 112 of the elongate body member 110 to expose the distal tips 122, 124 of the needles 116, 118. As shown in FIG. 5, the cover 128 is shown partially removed, but extends proximally along the elongate body member 110 beyond the mechanism 120. The cover 128 includes notches or other cutouts to accommodate the geometry of the mechanism 120 and any optional features. In addition, the cover 128 can include a thumb screw 129 that passes through a wall of the cover 128 and frictionally engages the elongate body member 110. The thumbscrew 129 can be loosened to allow the cover 128 to be retracted or extended relative to the distal end 112 of the elongate body member 110. The thumbscrew 129 can be tightened to maintain the cover 128 in its retracted or extended position.

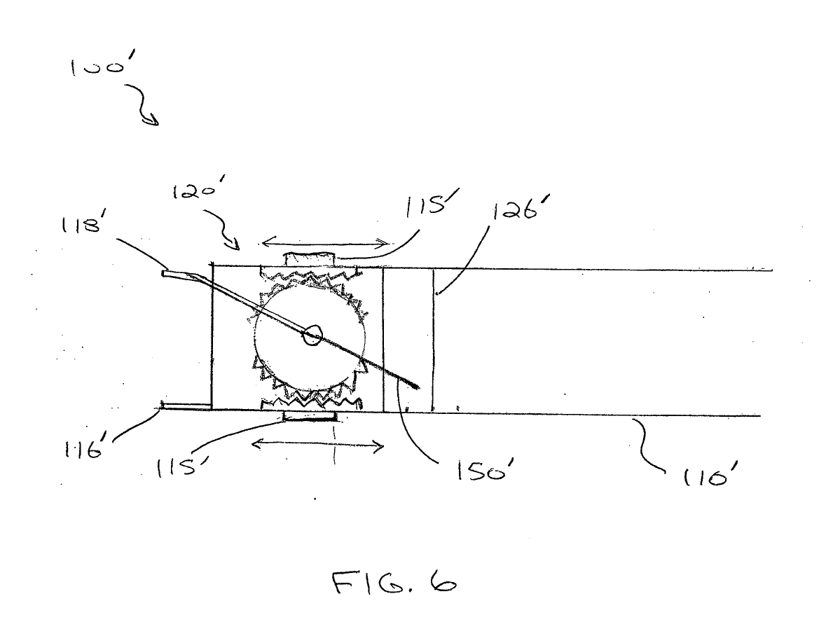

FIG. 6 depicts an alternative embodiment of the instrument of FIGS. 4 and 5 with an alternative mechanism 120′ for displacing the needles 118, 150. The mechanism 120′ includes at least one thumb slide mechanism 115′ disposed on the elongate body member 110′. In the embodiment shown, the mechanism 120′ includes two thumb slide mechanisms 115′, one disposed on each side of the elongate body member 110′. Generally, the thumb slide mechanisms 115′ include teeth 149″ that mesh with teeth 146′ at least partially disposed along the circumference of the wheel 140′ (see FIG. 7A). Linear movement of the thumb slide mechanism 115′ causes rotational movement of the wheel 140′, thus moving the indicator needle 150′ and the second needle 118′ relative to the first needle 116′. The thumb slide mechanisms 115′ are shown in greater detail in FIGS. 7A-7C.

As shown in FIGS. 7A-7C, the thumb slide mechanism 115′ includes a thumb slide portion 117′ and a rack portion 119′ interconnected by a stem portion 123′. The thumb slide portion 117′ is disposed proximate the exterior of the elongate body member 110′ and includes striations for facilitating sliding of the thumb slide portion 117′ by the user's thumb or finger. The rack portion 119′ is disposed proximate the interior surface of the elongate body member 110′ and oriented such that the teeth 149′ substantially fully engage the teeth 146′ disposed on the circumference of the wheel 140′. The stem portion 123′ passes though a slot 121′ extending through the wall of the elongate body member 110′.

The width of the slot 121′ is sized to have a minimum clearance between the sides of the stem portion 123′ and the edges of the slot 121′. The length of the slot 121′ generally corresponds to the linear distance the thumb slide mechanism 115′ moves to drive the second needle 118′ through its full range of motion, i.e., the length of the slot 121′ should accommodate the full linear travel of the thumb slide mechanism 115′ necessary to move the needle 118′ through it full range of motion. In one embodiment, the thumb slide portion 117′ is about 1 cm (0.4 inches) in length, about 3 mm (0.12 inches) in height, and about 3 mm (0.12 inches) in width; the rack portion 119′ is about 2 cm (0.8 inches) in length, about 3 mm (0.12 inches) in height, and about 3 mm (0.12 inches) in width. The stem portion 123′ is sized to provide sufficient spacing between the thumb slide portion 117′ and the rack portion 119′ to accommodate the wall thickness of the elongate body member 110′, such that the thumb slide mechanism 115′ is able to slide with minimal frictional resistance. The minimal frictional resistance should, however, be sufficient to prevent the thumb slide mechanism 115′ from moving inadvertently. Alternatively, the frictional resistance between the thumbwheel 140′ and elongate body member 110′, as described hereinabove, may be sufficient to prevent inadvertent movement of the needles 118′, 150′. The actual dimensions of the thumb slide mechanism components will vary to suit a particular application.

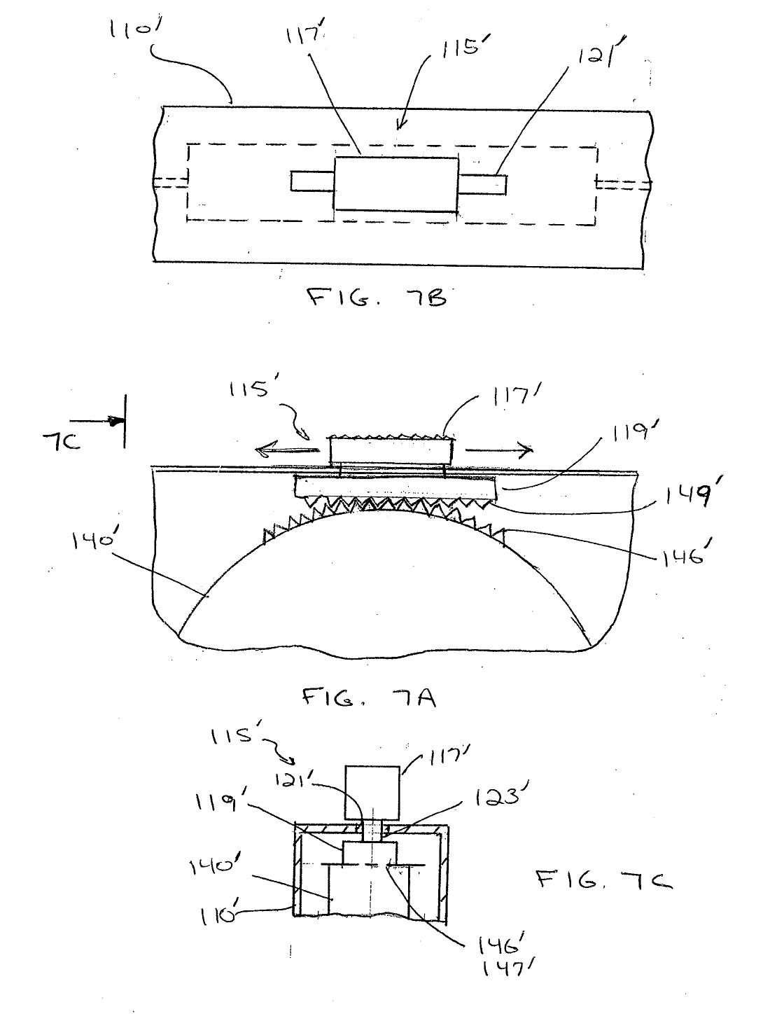

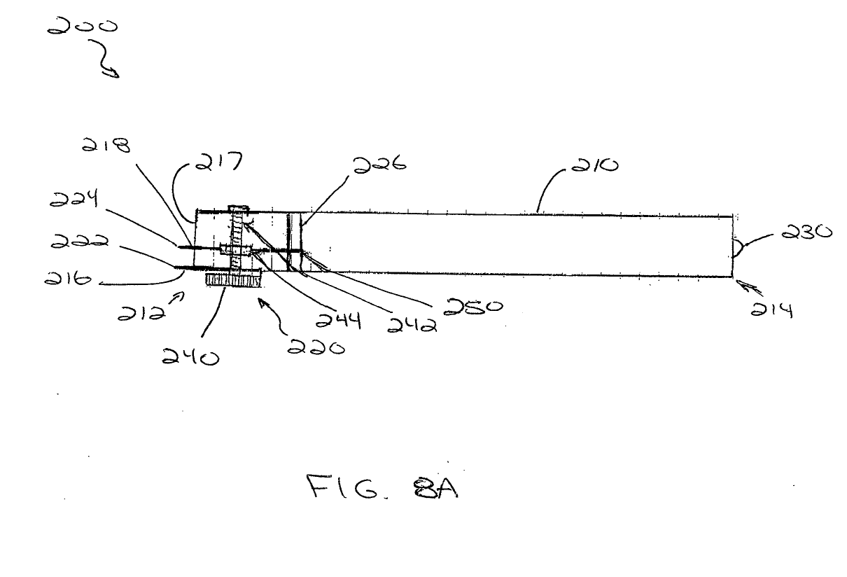

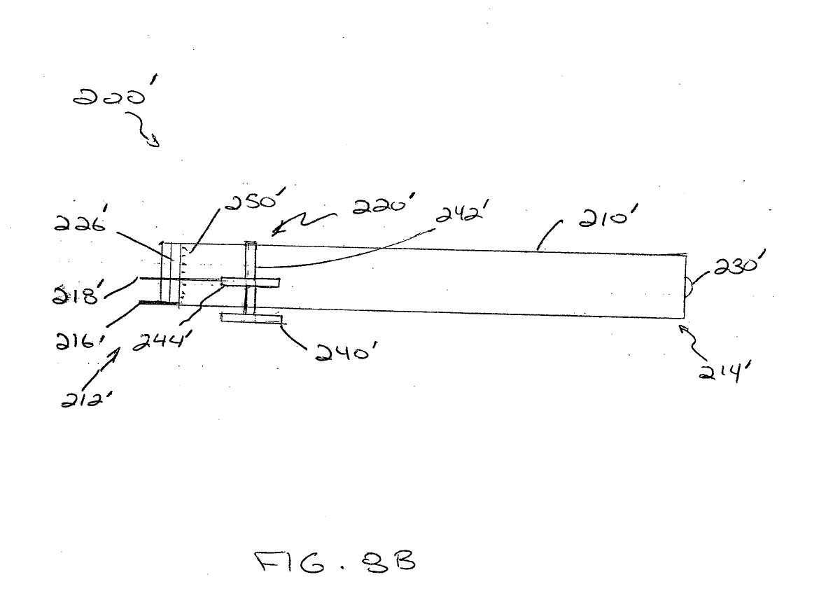

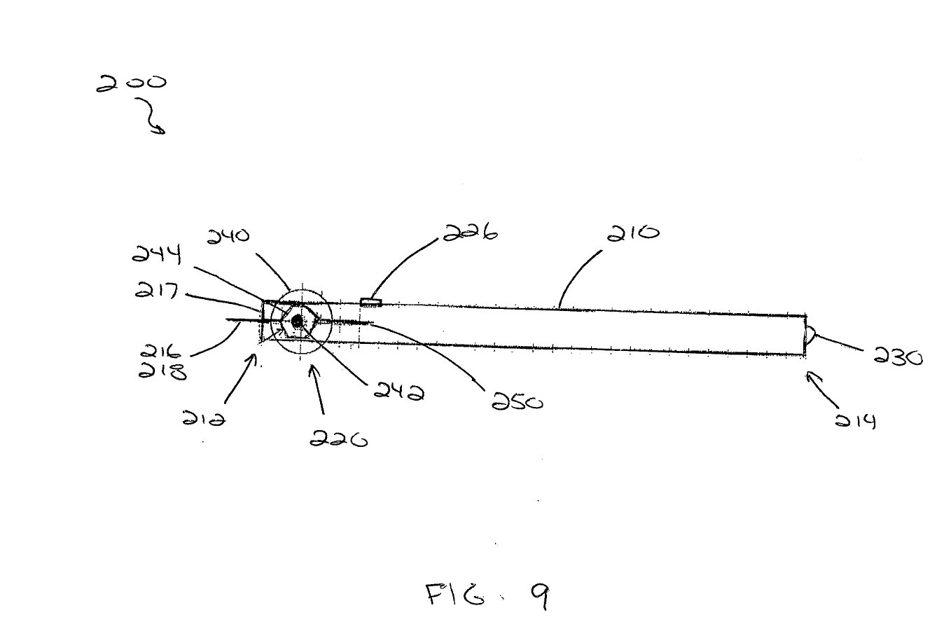

FIGS. 8A and 9 depict another alternative embodiment of an instrument 200 in accordance with the invention. The instrument 200 is similar to those previously described and includes an elongate body member 210, a first needle 216, a second needle 218, a mechanism 220, and a gauge 226. The first needle 216 is fixedly attached to the distal end 212 of the elongate body member 210 and the second needle 218 is coupled to the mechanism 220 and movable relative to the first needle 216. The instrument 200 can also include the optional features described hereinabove, for example an illumination source 230 located on the proximal end 214 of the elongate body member 210.

The mechanism 220 depicted in FIGS. 8A and 9 utilizes a worm gear arrangement to linearly displace the second needle 218 relative to the first needle 216. The mechanism 220 includes a thumbwheel 240 located on one side of the elongate body member 210. The thumbwheel 240 can be located on the left side, right side, or both of the elongate body member 210 relative to the gauge 226. In one embodiment, the thumbwheel 240 is about 1.5 cm (0.6 inches) in diameter and includes small transverse grooves inscribed on its circumferential surface for ease of turning. Attached to the center of the thumbwheel 240 is a threaded axle 242 that passes through the elongate body member 210 and is attached to the opposite side of the elongate body member 210. The axle 242 is mechanically coupled to the elongate body member 210 to allow the axle 242 to rotate freely.

Located along the axle 242 within the elongate body member 210 is a threaded nut 244 threadedly engaged with the axle 242. The nut 244 shown in FIGS. 8A and 9 is hexagonal in shape; however, any shape is contemplated and within the scope of the invention, as long as the nut 244 can threadedly engage the axle 242. The nut 244 will move along the axle 242 as the thumbwheel 240 is rotated. In one embodiment, the nut 244 is about 1 cm (0.4 inches) in diameter; however, the nut 244 may be longer in certain embodiments to provide better support to the needles. The size and shape of an internal cavity of the elongate body member 210 is such that the nut 244 is free to move along the length of the axle 242, but is constrained against rotating with the axle 242 as the thumbwheel 240 is rotated. Therefore, the first and second needles 216, 218 will remain in a common plane. Alternatively or additionally, an end surface 217 of the elongate body member 210 may include a slot or a slotted insert could be inserted into the distal end 212 of the elongate body member 210 to support the needles 216, 218 in a common plane and counteract lateral and up/down stresses arising on the needles 216, 218.

The second needle 218 is attached to the nut 244 and moves along the axle 242 as the thumbwheel 240 is rotated. Also attached to the nut 244 is an indicator needle 250 that also moves along the axle 242 as the thumbwheel 240 is rotated. The second and indicator needles 218, 250 can be bonded or otherwise mechanically coupled to the nut 244. In one embodiment, both needles 218, 250 are about 1.5 cm (0.6 inches) in length and about 1 mm (0.04 inches) in diameter.

In one embodiment, the thread pitch of the threaded axle 242 and nut 244 is about 2 mm (0.08 inches), which translates into one full turn of the thumb wheel 240 equals about 2 mm (0.08 inches) of linear movement of the second and indicator needles 218, 250. In this embodiment, the one full turn of the thumb wheel 240 will move the second needle 218 to a next sensory testing increment (i.e., from 4 mm to 6 mm). In an embodiment of the instrument 200 where the maximum distance between the first and second needles 216, 218 is 12 mm (0.5 inches), the second and indicator needles 218, 250 will travel across the entire scale in about six full turns.

The first needle 216 is fixedly coupled to the distal end 212 of the elongate body member 210. The first needle 216 may be bonded to or otherwise mechanically coupled to an inner wall of the elongate body member 210. The first needle 216 is fixed to the elongate body member 210 to prevent movement of the first needle 216 during use of the instrument 220 (i.e., the first needle 216 is stationary while the second needle 218 moves relative to the fixed first needle 216); however, the first needle 216 may be removable from the elongate body member 210 for replacement with a needle having a different distal tip 222 (i.e., a blunt tip vs. a sharp tip or a linear needle vs. a curved needle).

The specific dimensions of the needles 216, 218, 250 will vary to suit a particular configuration of the instrument 200. For example, the length of the needles 218, 250 will depend on the location of the mechanism 220 relative to the distal end 212 of the instrument 200 and the size of the components (e.g., a standard vs. elongated nut 244). Generally, the mechanism 220 is located proximate the distal end 212 of the elongate body member 210 and the first and second needles 216, 218 should be configured to extend beyond the distal end 212 of the elongate body member 210 by about 4 mm to about 8 mm (0.16-0.32 inches). The configuration of the indicator needle 250 will depend on the placement of the gauge 226 relative to the mechanism 220. As previously described, the distal end 212 of the instrument may be transparent to facilitate viewing of the indicator needle 250. In one embodiment, the gauge 226 is located about 1.5 cm (0.6 inches) proximal to the axle 242. The gauge 226 can include markings as previously described.

FIG. 8B depicts an alternative version of the instrument of FIG. 8A. The instrument 200′ depicted in FIG. 8B is substantially similar to the instrument 200 of FIG. 8A, except the gauge 226′ and scale 250′ are disposed distally of the mechanism 220′, i.e., at the distal end 212′ of the instrument 200′. In such an arrangement, the separate indicator needle 250 is not required, as the second needle 218′ also acts as the indicator needle relative to the gauge 226′.

FIGS. 10A and 11 depict yet another alternative embodiment of an instrument 300 in accordance with the invention. The instrument 300 is similar to those previously described. Generally, the instrument 300 includes an elongate body member 310, a first needle 316, a second needle 318, a mechanism 320, and a gauge 326. The first and second needles 316, 318 are coupled to the mechanism 320 and movable relative to one another. The instrument 300 can also include the optional features described hereinabove, for example, a cover, a pocket clip, an illumination source, a power source, and a scale.

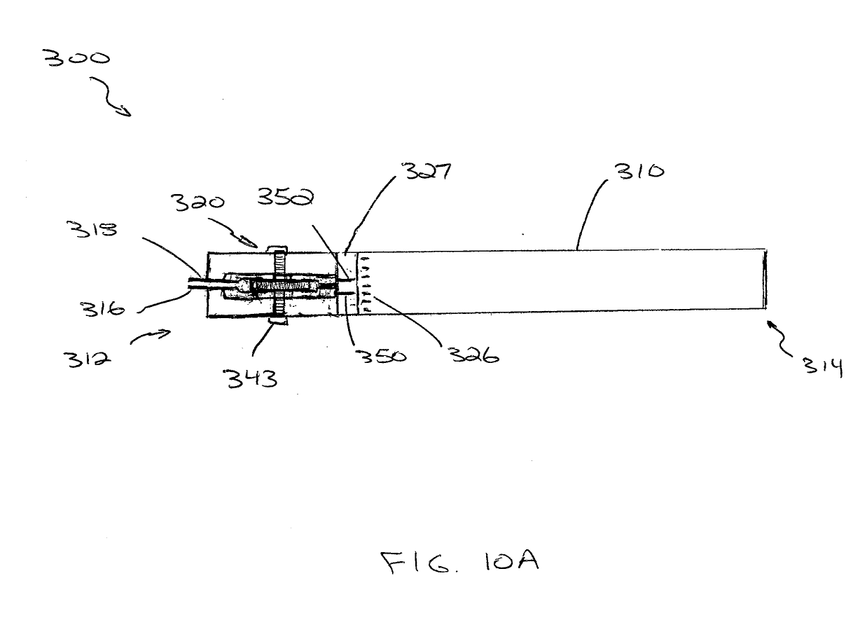

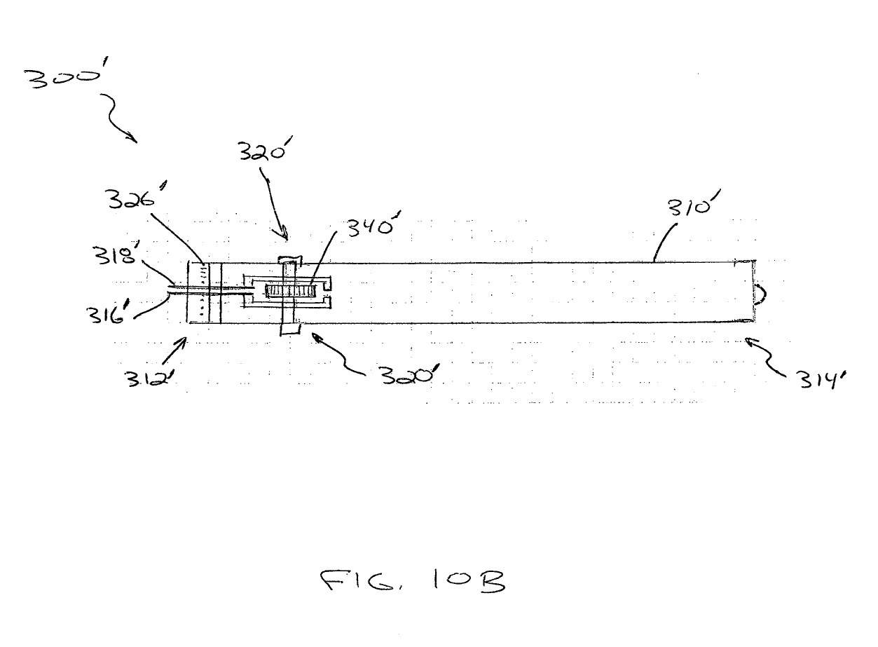

The mechanism 320 and its related components are shown in greater detail in FIGS. 12 and 13. Generally, the mechanism 320 includes a thumbwheel 340 centrally located through the elongate body member 310. Attached to the thumbwheel 340 are two axles 342, 342′. Each axle 342, 342′ extends from an opposing face of the thumbwheel 340 through the elongate body member 310. One end of each axle 342, 342′ is bonded to or otherwise mechanically fixed to the thumbwheel 340 so that the axles 342, 342′ turn with the thumbwheel 340. The other ends of the axles 342, 342′ are mechanically coupled to the elongate body member 310 to allow the axles 342, 342′ to rotate freely when the thumbwheel 340 is rotated. For example, the axles 342, 342′ could be secured to the elongate body member 310 with a threaded collar or secured with a pin. In one embodiment, the thumbwheel 340 is about 1.5 cm (0.6 inches) in diameter and about 3 mm (0.12 inches) in width. The thumbwheel 340 projects about 3 mm (0.12 inches) beyond the front and rear surfaces 311, 313 of the elongate body member 310 to allow thumb or finger turning. In one embodiment, each axle 342 is threaded and about 3 mm (0.12 inches) in diameter and about 0.6 cm (0.25 inches) in length. The thumbwheel and axle assembly is about 1.5 cm (0.6 inches) in overall length. In one embodiment, each axle is mounted on a circular mounting 343 that allows frictionless rotation, for example a radial bearing.

The mechanism 320 further includes two needle support bars 344, 345, with one bar disposed on each side of the thumbwheel 340. The bars 344, 345 threadedly engage their corresponding axle 342, 342′ on each side of the thumbwheel 340. One of the bars 345 is depicted in FIGS. 13A and 13B. Each bar 344, 345 moves along its respective axle 342, 342′ when the thumbwheel 340 is rotated. Specifically, rotating the thumbwheel 340 causes the axles 342, 342′ to rotate, which in turn forces the bars 344, 345 to ride along their respective axle 342, 342′ and, thereby, sets the distance between the two needles 316, 318 relative to one another and the longitudinal center of the elongate body member 310. This mechanism arrangement allows the needles 316, 318 to be moved concomitantly and symmetrically along their respective axles 342, 342′. The bars 344, 345 fit into an internal cavity 311 of the elongate body member 310 such that the bars 344, 345 are free to move laterally, but are prevented from rotating with their respective axle 342, 342′.

Referring to FIGS. 13A and 13B, in one embodiment, the bars 344, 345 measure about 3 cm (1.2 inches) in length and about 5 mm (0.2 inches) in thickness. The outside surface 349 of the bar is substantially planar. The inside surface 347 has a central linear depression 351 that measures, in one embodiment, about 2 cm (0.8 inches) in length. The thickness of the bar 344, 345 at this point is about 2 mm (0.08 inches). The bars 344, 345 include raised ends 353 that project toward the thumbwheel 340. Medial surfaces 355 of the raised ends are offset (d₂) from the central linear depression by about 2 mm (0.8 inches). The medial surfaces 355 are substantially planar and, in one embodiment, measure about 5 mm (0.2 inches) in length. In addition, the bars 344, 345 include a threaded through hole 357 through which the axles 342, 342′ pass and engage their corresponding bar 344, 345. In one embodiment, the thread pitch of the threaded axles 342, 342′ and the bars 344, 345 is about 1 mm (0.04 inches). One full rotation of the thumb wheel 340 causes each bar 344, 345 and corresponding needle 316, 318 to simultaneously move about 1 mm (0.04 inches). Approximately five full turns of the thumb wheel 340 will translate into moving the needles 316, 318 through a full range of travel of about 1 cm (0.4 inches).

In one embodiment, the medial surfaces 355 serve as a mounting surface for the needles 316, 318, 350. The needles 316, 318, 350, 352 may be bonded or otherwise mechanically attached to the medial surfaces 355. Alternatively, the raised ends 353 may include a clearance hole 319 formed in an end face of the raised ends 353 for receiving the needles 316, 318, 350. The needles 316, 318, 350 can be force fit into the hole 319 and/or bonded therein. The hole 319 or other mechanical means for fastening the needles 316, 318, 350, 352 to the bars 344, 345 are configured such that the distance (d₁) between the distal tips of the needles when in their closed state is a maximum of 2 mm (0.08 inches), and the distance (d₁) between the distal tips of the needles when in their open state is a minimum of about 1 cm (0.4 inches).

The length and diameter of each needle 316, 318, 350, 352 may vary to suit a particular application and will depend at least in part on the location of the mechanism 320 relative to the distal end 312 of the elongate body member 310 and the gauge 326. The needles 316, 318 need to extend beyond the distal end 312 of the elongate body member 310 far enough to suitably interact with the appendage to be tested. The indicator needles 350, 352 need to extend far enough beyond the mechanism to interact with the gauge 326. In one embodiment, the sensory needles 316, 318 are about 1 mm (0.04 inches) in diameter and about 1 cm (0.4 inches) in length, with a blunt tip. In one embodiment, the indicator needles 350, 352 located opposite the sensory needles 316, 318 are about 1 mm (0.04 inches) in diameter and about 5 mm (0.2 inches) in length. The indicator needles 350, 352 interface with the gauge 326 to show the distance between the distal tips 322, 324 of the sensory needles 316, 318.

In one embodiment, the gauge 326 includes a translucent window 327 oriented transversely to the elongate body member 310. The window 327 may be about 1.5 cm (0.6 inches) in length and about 4 mm (0.16 inches) in width. The indicator needles 350, 350′ are visible through the translucent window 327. The gauge 326 also includes a scale with markings on each side of the midline of the elongate body member 310 progressing from 2 mm to 1 cm (i.e., 10-8-6-4-2-2-4-6-8-10).

FIG. 10B depicts an alternative version of the instrument of FIG. 10A. The instrument 300′ depicted in FIG. 10B is substantially similar to the instrument 300 of FIG. 10A, except the gauge 326′ is disposed distally of the mechanism 320′, i.e., at the distal end 312′ of the instrument 300′. In such an arrangement, the separate indicator needles 350, 352 are not required, as the sensory needles 316′, 318′ also act as the indicator needles relative to the gauge 326′.

FIGS. 14-16 depict another alternative embodiment of a two-point discriminator instrument 400 in accordance with the invention. The instrument 400 is similar to those previously described. Generally, the instrument 400 includes an elongate body member 410, a first needle 416, a second needle 418, a mechanism 420, and a gauge 426. The first and second needles 416, 418 are coupled to the mechanism 420 and movable relative to one another. The mechanism 420 differs from the previous embodiments in that a bifurcated beam and splitter arrangement replaces the thumbwheel assembly. The instrument 400 can also include the optional features described hereinabove, for example, a cover, a pocket clip, an illumination source, a power source, and a scale.

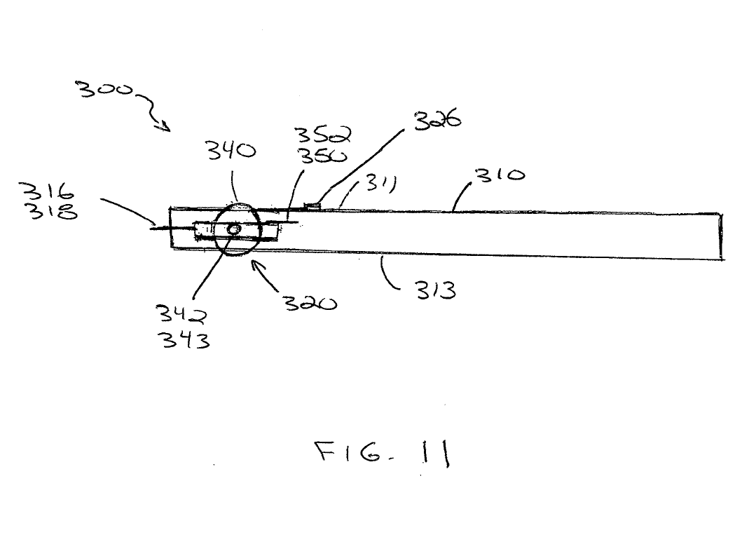

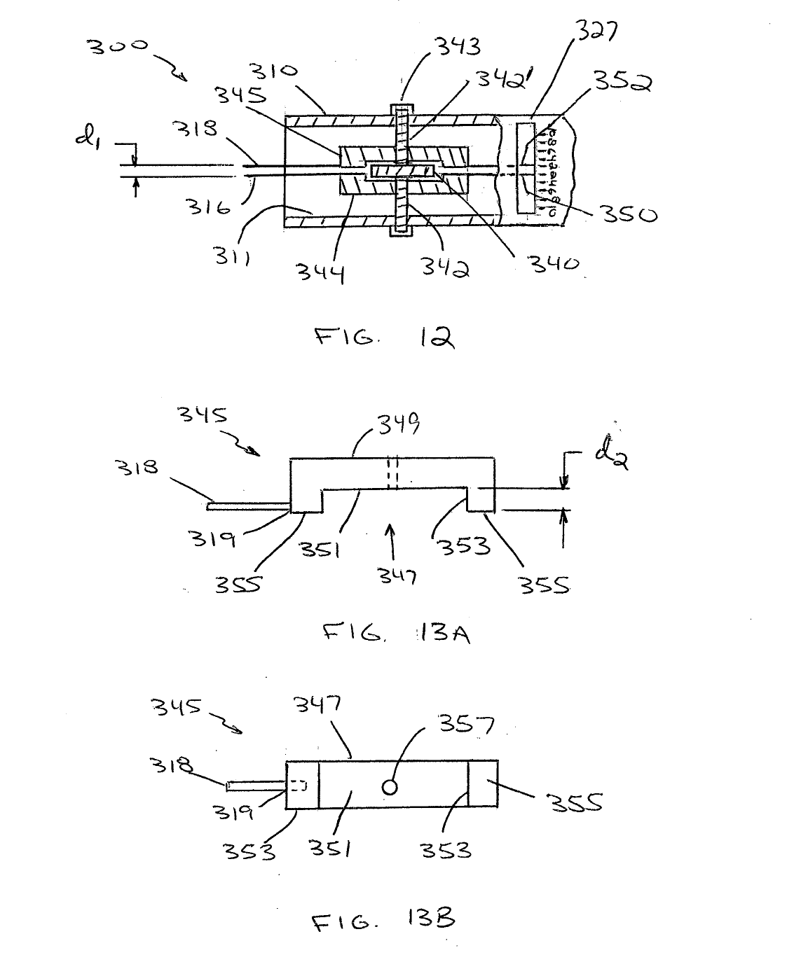

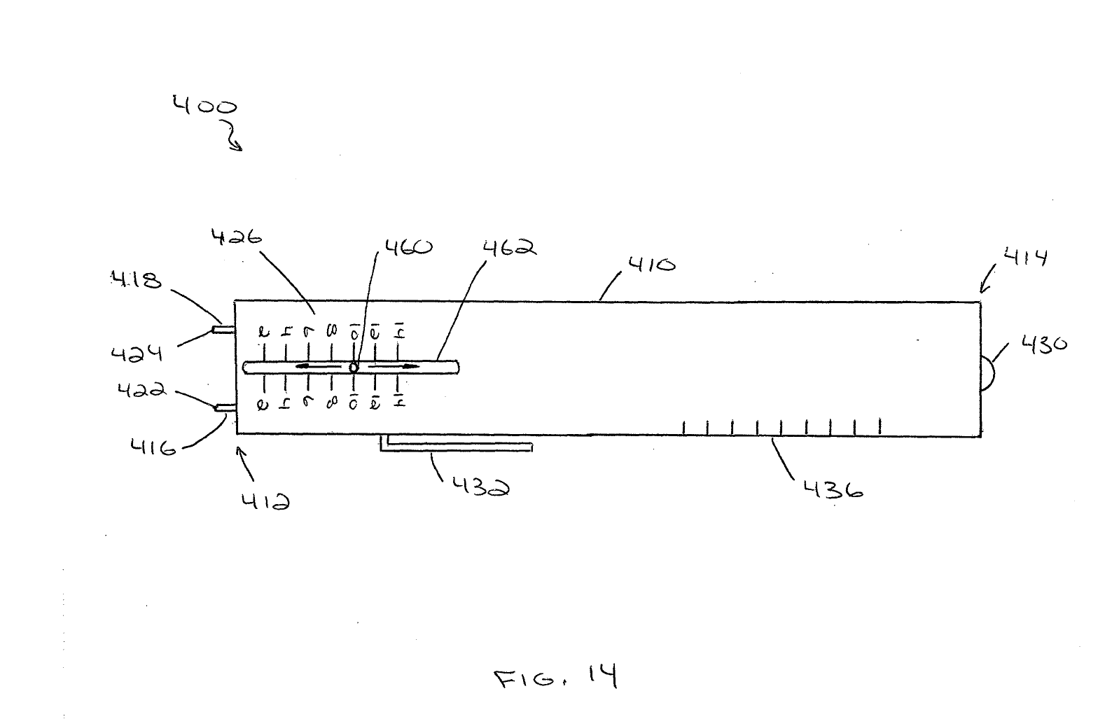

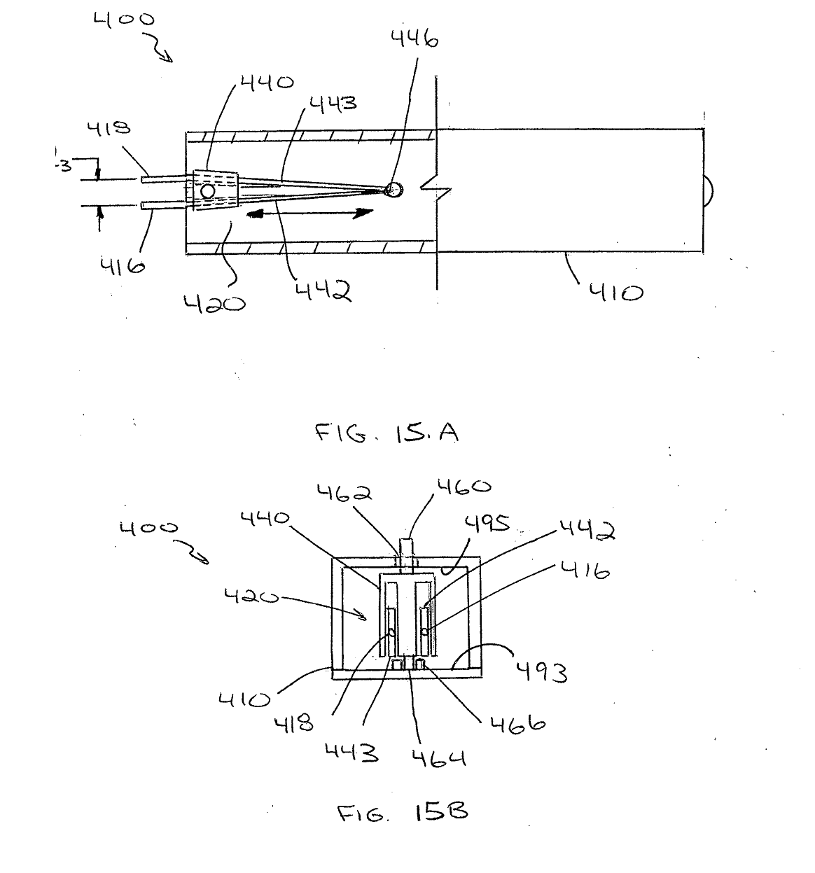

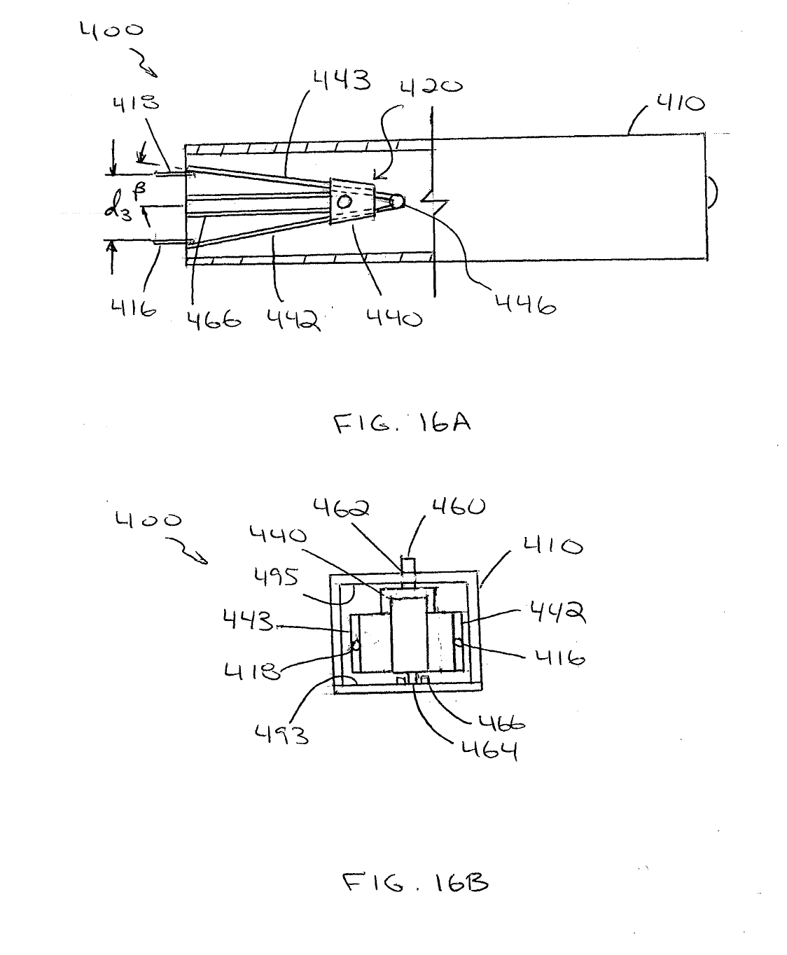

The instrument 400 is shown in a “closed” configuration (i.e., the needles 416, 418 are located about 2 mm apart) in FIGS. 15A and 15B and in an “open” configuration (i.e., the needles 416, 418 are forced apart) in FIGS. 16A and 16B. The mechanism 420 includes a wedge shaped cam or beam splitter 440 (see FIG. 17) and two bands 442, 443 coupled together to form the bifurcated beam. The beam is coupled to the elongate body member 410 proximal to the distal end 414 thereof via a pin 446 or directly to the elongate body member 410. The two bands 442, 443 are configured for interaction with the splitter 440. The first and second needles 416, 418 are coupled to the distal ends of their respective band 442, 443. The needles 416, 418 are attached to the bands 442, 443 via wedge shaped spacers 480, 482 (FIG. 18) that maintain the substantially longitudinal orientation of the needles 416, 418 as the bands 442, 443 move between the closed and open configurations. The needles 416, 418 can be attached to the spacers 480, 482 by bonding or otherwise mechanically coupled thereto.

In one embodiment, the pin 446, which acts as a pivot point for each of the bands 442, 443, is located about 3.5 cm (1.4 inches) from the distal end 412 of the elongate body member 410. In this embodiment, each band 442, 443 has an angular range of travel (β)(see FIG. 16A) of between about 1.5 degrees and about 10 degrees, which translates to a linear displacement of one of the needles 416, 418 of about 1 mm (0.04 inches) to about 6 mm (0.25 inches).

The splitter 440 has a rail 464 projecting from a bottom surfaced thereof that engages and rides in a track 466 disposed on the inside bottom surface 493 of the elongate body member 410. Disposed on a top surface of the splitter 440 is a handle 460 that extends through a slot or track 462 in a top surface 495 of the elongate body member 410. The handle 460 acts as a linear actuator for manually positioning the splitter 440 along the length of the elongate body member 410 with the user's thumb or finger. The handle 460 interacts with the track 462 to prevent inadvertent lateral movement of the splitter 440 and with the gauge 426 disposed on either side of the track 462 to indicate the distance (d₃) between the needles 416, 418. In one embodiment, the gauge 426 includes markings from 2 mm to 12 mm on each side of the track 462. The splitter 440 “floats” within the elongate body member 410 with the tracks 462, 466 constraining the movement of the splitter 440. Movement of the handle 460 causes movement of the splitter 440 and in turn concomitant and symmetrical movement of the two needles 416, 418. For example, the splitter 440 and bands 442, 443 are configured such that about 2 mm (0.08 inches) movement of the handle 460 causes the distance between the needles 416, 418 to change by about 2 mm (0.08 inches).

Referring to FIGS. 17A and 17B, the splitter 440 has a generally triangular or wedge shaped body 470 with the handle 460 projecting upwardly therefrom. Generally, the angle of the wedge will vary to suit a particular application and will depend, in part on the overall size of the instrument and the range of travel of the splitter 440 within the instrument 400. The splitter 440 also includes two arms 472, 474 that extend outwardly and down from a top surface of the splitter 440, thereby defining a substantially parallel gap 476 between the arms 472, 474 and the cam body 470. The gaps 476 are configured to receive the bands 442, 443 carrying the needles 416, 418 so that the arms 472, 474 guide the bands 442, 442 as the splitter 440 is moved linearly.

In one embodiment, each band 442, 443 is made from spring steel and measures about 3.5 cm (1.4 inches) in length, about 4 mm (0.16 inches) in height, and about 2 mm (0.08 inches) in thickness. The bands 442, 443 can be attached to the pin 446 at their proximal ends. The pin 446 can extend from the top or bottom inner surfaces 495, 493 of the elongate body member 410. Alternatively, the proximal ends of the bands 442, 443 can be directly joined to the bottom inner surface 493 of the elongate body member 410, for example by bonding.

The bottom track 466 runs along the inside bottom surface 493 from the point where the proximal ends of the bands 442, 443 are attached to proximate the distal end 412 of the elongate body member 410. In one embodiment, the track 466 runs about 3 cm (1.2 inches). The length of the track 466 will be sized such that the distance of travel of the splitter 440 in the track 466 corresponds to a range of distance between the first and second needles 416, 418 of about 2 mm (0.08 inches) to about 12 mm (0.5 inches). The track 466 can be formed by attaching two side rails to the inner bottom surface 493 of the elongate body member 410 or could be formed in the inner bottom surface 493 by machining or during a molding process. The width of the track 466 is slightly larger than the width of the rail 464 to allow the splitter 440 to move freely along the track 466. Alternatively, the width of the track 466 could be sized to provide a slight friction fit so that the splitter 440, and by extension the needles 416, 418, maintain their position until the user applies a force to the handle 460.

The position of the bands 442, 443 are further controlled by the arms 472, 474 on the splitter 440, as the bands 442, 443 are kept in alignment by the arms 472, 474. Attached to approximately the vertical midline of each band 442, 443 are the wedge shape spacers 480, 482. Each spacer 480, 482 may include a receptacle for receiving one of the needles 416, 418. The spacers 480, 482 offset the needles 416, 418 from the band 442, 443, which are oriented at an angle to the elongate body member 410, such that the needles 416, 418 are oriented substantially parallel to the longitudinal axis of the elongate body member 410 and each other. In one embodiment, the needles 416, 418 are about 1.0 cm (0.4 inches) in length and about 1 mm (0.04 inches) in diameter.

FIG. 18 depicts an exploded end view of the instrument 400 of FIG. 14; however, the basic principles of assembly can be applied to any of the embodiments described herein. As shown in FIG. 18, the elongate body member 410 includes a top housing 497 having a substantially rectangular cross-sectional shape and an open bottom surface. The elongate body member 410 also includes a separate bottom housing 490 having a substantially planar shape. The internal components of the mechanism 420 are located substantially within the top housing 497. The bottom housing 490 snap fits or can be bonded to the top housing 497 to secure the various components of the mechanism 420 together. The bottom housing 490 can include a stepped portion 492 that is configured to engage the side walls of the top housing 497. The stepped portion 492 may provide frictional engagement with the top housing 497 to assist in securing the two housings 490, 497.

As previously described, the track 466 is located on the inner bottom surface 493 of the bottom housing and the slot 462 is formed in the top surface of the top housing 497. In one method of assembling the instrument 400, the bifurcated beam assembly 499 (i.e., the bands 442, 443, pin 446, spacers 480, 482, and needles 416, 418 can be fixed to the inner bottom surface 493 of the bottom housing 490 via the pin 446. The splitter 440 can then be positioned over the beam assembly 499 by directing the bands 442, 443 into their respective gap 476 and fitting the rail 464 within the track 466. The top housing 497 can then be place over the mechanism 420, with the handle 460 extending through the slot 462. The top and bottom housings can then be secured together as previously described. Once assembled, the splitter 440 can be moved linearly along a length of the instrument 400 via the handle 460 to move the needles 416, 418 towards and away from one another, thereby varying the distance between the needles 416, 418.

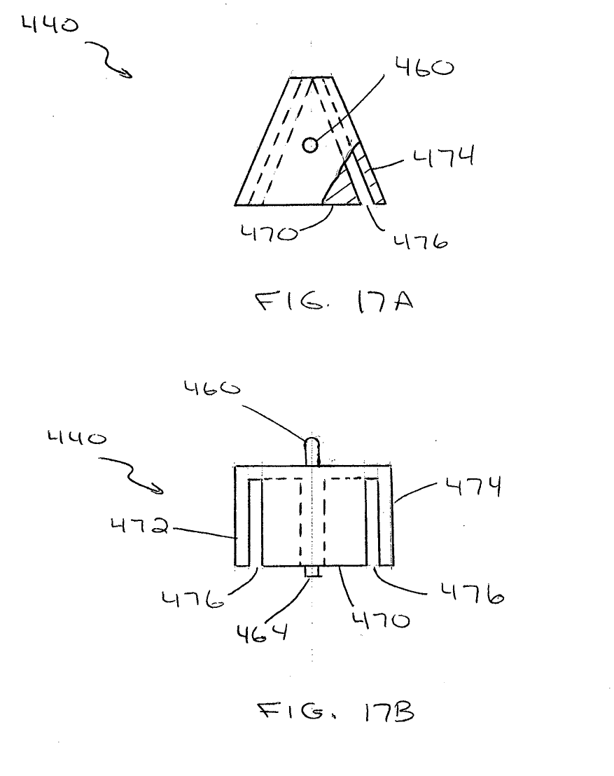

FIGS. 19 and 20 depict alternative versions of the instrument of FIG. 14. The instruments 400′, 400″ depicted in FIGS. 19 and 20 are substantially similar to the instrument 400 of FIG. 14, including using a similar bifurcated beam and splitter mechanism 420′ The instrument 400′ of FIG. 19 differs from the instrument 400 of FIG. 14 by relocating the gauge 426′ to the distal end 412′ of the instrument 400′ (similar to FIG. 10B). The instrument 400″ of FIG. 20 differs from the instrument 400 of FIG. 14 by using angled needles 416′, 418′.

As shown in FIG. 20, the needles 416″, 418″ are curved or gently angled downwardly from the top surface 417″ of the instrument 400″. Because the instrument in use is typically oriented at an angle to the pads of the fingers, orienting the needles at a similar angle can improve the end on contact of the needles with the pads of the fingers or other appendage. In one embodiment, the angle of orientation of the needles (a) is about 45 degrees to about 70 degrees. As also shown in FIG. 20, the distal points of the needles 416″, 418″ do not extend beyond a bottom surface 419″ of the instrument 400″, which would allow a cover as previously described to be used with the instrument 400″. In such an arrangement, the attachment points of the needles 416″, 418″ relative to the bands 442″, 443″ may be adjusted to facilitate and/or compensate for the angle or curve of the needles 416″, 418″.

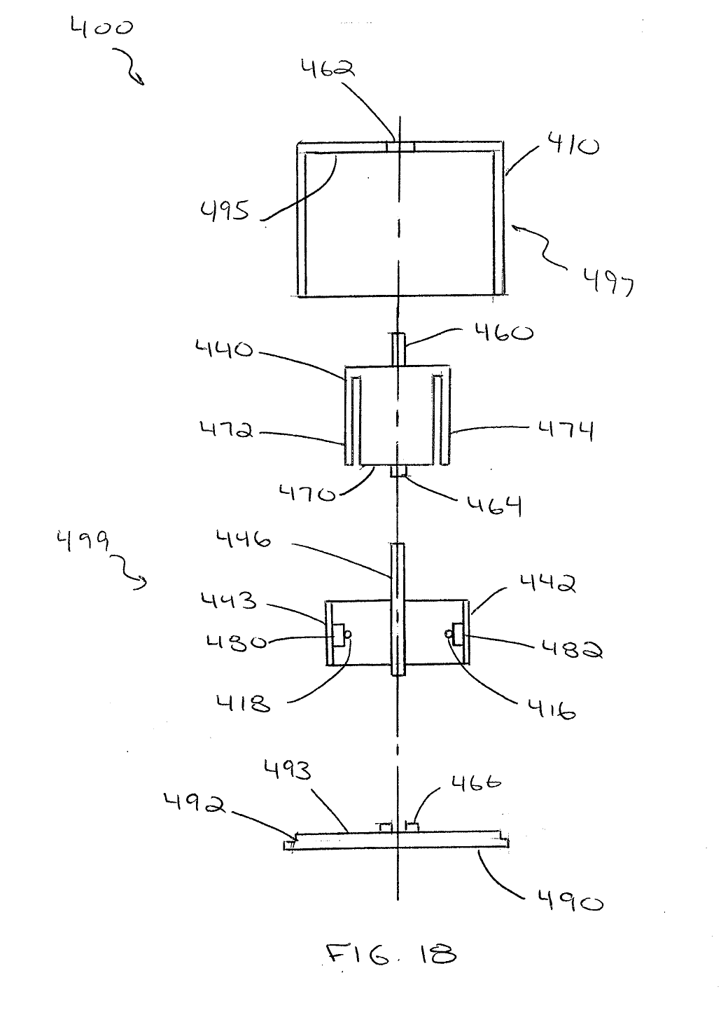

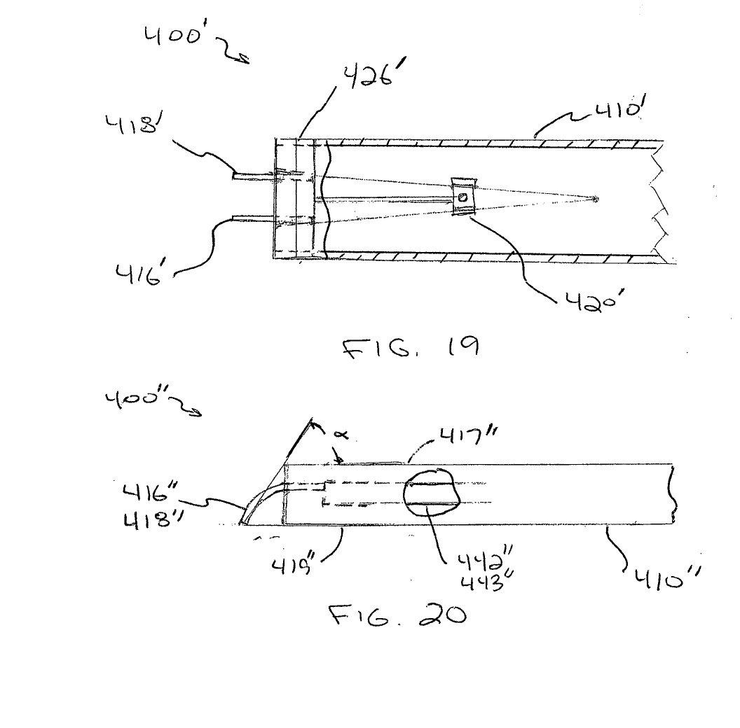

FIGS. 21A-21C depict an alternative embodiment of a two-point discriminator instrument 500 in accordance with the invention. The alternative instrument 500 is similar to the instruments 1 described hereinabove and includes a goniometer 501 coupled to the elongate body member 510. The goniometer 501 is typically located at the proximate end 514 of the instrument 500, opposite the two-point discriminator mechanism. Generally, the goniometer 501 operates via a double ended rod that swivels on an axle and a scale that measures degrees of angulation. An accurate measurement of angles up to 180 degrees can be obtained with this device.

There are situations in hand surgery practice, as well as in other surgical and medical specialties, where a measurement of bony angulation (in the case of fractures) or measuring the range of joint motion is necessary. This is true in the office, emergency room and clinic. Having a goniometer built into the present instrument 500 would extend the use of the instrument to those situations. In addition, a two point discriminator with a built-in goniometer eliminates the need to carry a separate instrument.

With reference to FIGS. 21A-21C, the instrument 500 includes an opening or pocket 502 formed in the elongate body member 510. In one embodiment, the pocket 502 is a recess formed in the proximate end 514 of the elongate body 510, for example, the pocket 502 can be located where the upper surface and side surface of the elongate body member meet. The pocket 502 may disposed on the side or end opposite the optional pocket clip. In one embodiment, the pocket is about 4 cm (1.5 inches) in length and about 4 mm (0.15 inches) in width and depth. The exact measurements of the pocket 502 and associated components of the goniometer 501 will vary to suit a particular application.

The pocket 502 houses an indicator rod 506 and associated operating mechanism, which will be described in greater detail later. The goniometer 501 also includes a semi-circular scale 504 disposed on the top surface of the elongate body member 510. The scale 504 may be inscribed or silk-screened onto the elongate body member 510; however, other methods of providing a scale on the body member 510 are contemplated and considered within the scope of the invention. The scale 504 includes markings from 0 degrees to 180 degrees. In one embodiment, the degree markings can be every 5 degrees with a larger marking every 45 degrees. The size, frequency, and shape of the markings may vary to suit a particular application and may also include LED indicators, as previously described with respect to the alternative versions of the instrument.

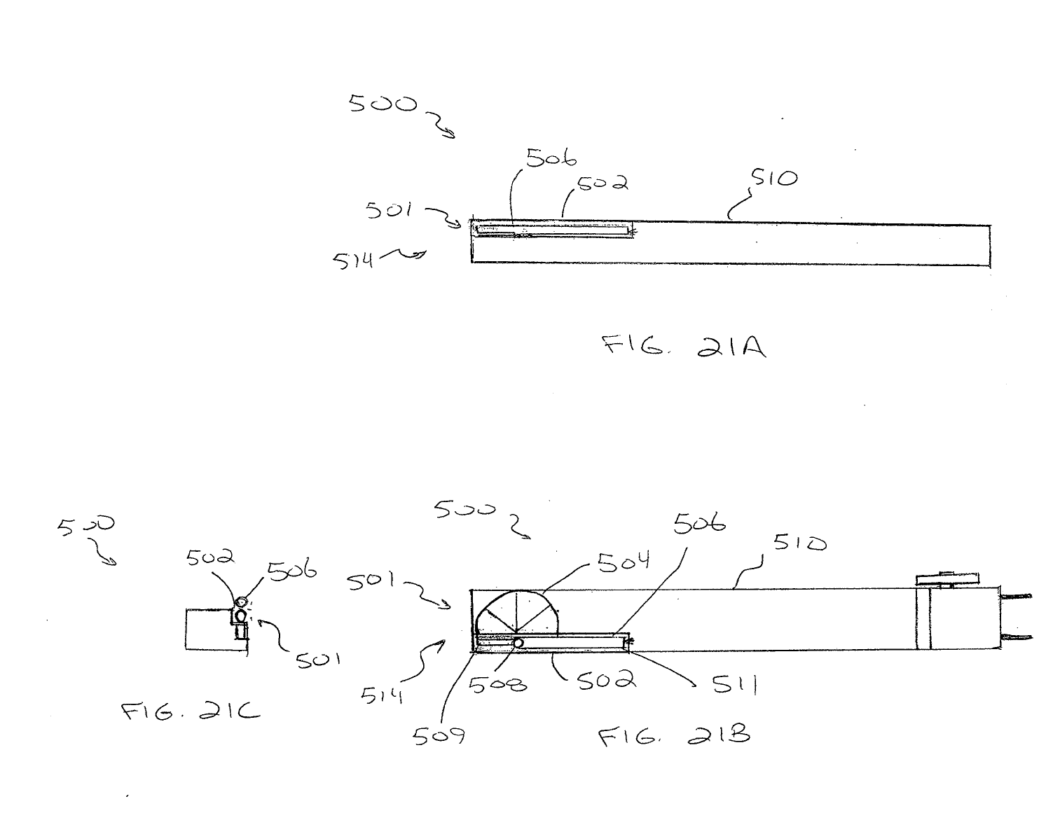

The rod 506 used for providing the angular indication in one embodiment is about 4 cm (1.5 inches) in overall length and about 2-3 mm (0.08-0.12 inches) in thickness or diameter. The rod 506 may be square with slightly rounded edges, round, or oval in cross section. A distal portion 507 of the rod 506 (approximately 1 to 1.2 cm (0.4-0.5 inches) in length) is aligned on the proximal surface of the housing and is the part of the rod 506 that is rotated over the scale 504 (see FIG. 22), thereby providing the indication of the degrees of angulation. The remainder of the rod 506, or the measuring portion 511 (approximately 3 cm (1.2 inches) in length in one embodiment), is seated within the pocket 502 when the goniometer 501 is not in use. This portion of the rod 506 is that which is placed along the object to be measured. The specific lengths and diameter of the rod 506 will vary and, in one embodiment, the distal end or “marking” portion 507 of the rod 506 may be of a different diameter than the longer “measuring” portion 511 of the rod 506 (see FIG. 22).

The operating mechanism of the goniometer 501 includes the rod 506 and a pin or axle 508 that couples the rod 506 to the elongate body member 510, but also allows the rod 506 to be slid, pivoted, or otherwise positioned out of the pocket 502 and into an operational configuration. The operational configuration allows the rod 506 to be rotated and provide the angular measurement of an object.

As shown in greater detail in FIGS. 22-24, the axle 508 is disposed within an opening located within the elongate body member 510 (see FIG. 23). The opening or hole 512, in one embodiment, is located about 1.1 cm (0.43 inches) from the proximate end 514 of the body member 510 and generally corresponds to the center point of the semi-circular scale 504. In one embodiment, the hole 512 is about 2-4 mm (0.08-0.15 inches) in diameter and the center axis of the hole 512 is generally parallel to the side wall of the elongate body member 510 (see FIG. 23). The hole 512 may include a tubular sleeve 513 disposed therein. In one embodiment, the sleeve 513 has a diameter of about 3-5 mm (0.12-0.2 inches) and is fixed within the hole 512. The sleeve 513 may provide a friction fit or a sliding fit to the axle 508 depending on the tolerance between the axle 508 and sleeve 513 and the materials thereof. Preferably, the axle 508 and sleeve 513 or opening 512 have a friction fit to prevent inadvertent movement of the rod 506.

The axle 508 may include a protuberance or other enlarged structure 517 on one end to slidingly secure the axle 508 within the hole 512. Alternatively, the axle 508 may be secured within the hole 512 with a pin and groove arrangement that allows the axle to slide within the hole 512, but not slide out of the hole 512. In one embodiment, the axle 508 (FIG. 23) can be joined to the rod 506 by either passing a distal portion of the axle 508 through the rod 506 or around the rod 506. Alternatively, the rod 506 and axle 508 can be joined by other mechanical means, for example a fastener, bonding, or welding. In one embodiment, the axle 508 measures about 4-6 mm (0.15-0.25 inches) in length and about 2-3 mm (0.08-0.12 inches) in diameter and fits inside the sleeve 513. At the end of the axle 508 inside the body member 510, a disk 517 measuring about 5-7 mm (0.2-0.28 inches) in diameter is attached to the axle 508. This arrangement of the axle 508 allows the rod 506 to be extended beyond the flat top surface of the two point discriminator when measuring angulation or retracted to below the surface when not in use. The disk 517 keeps the axle 508, and by extension the rod 506, from falling out.

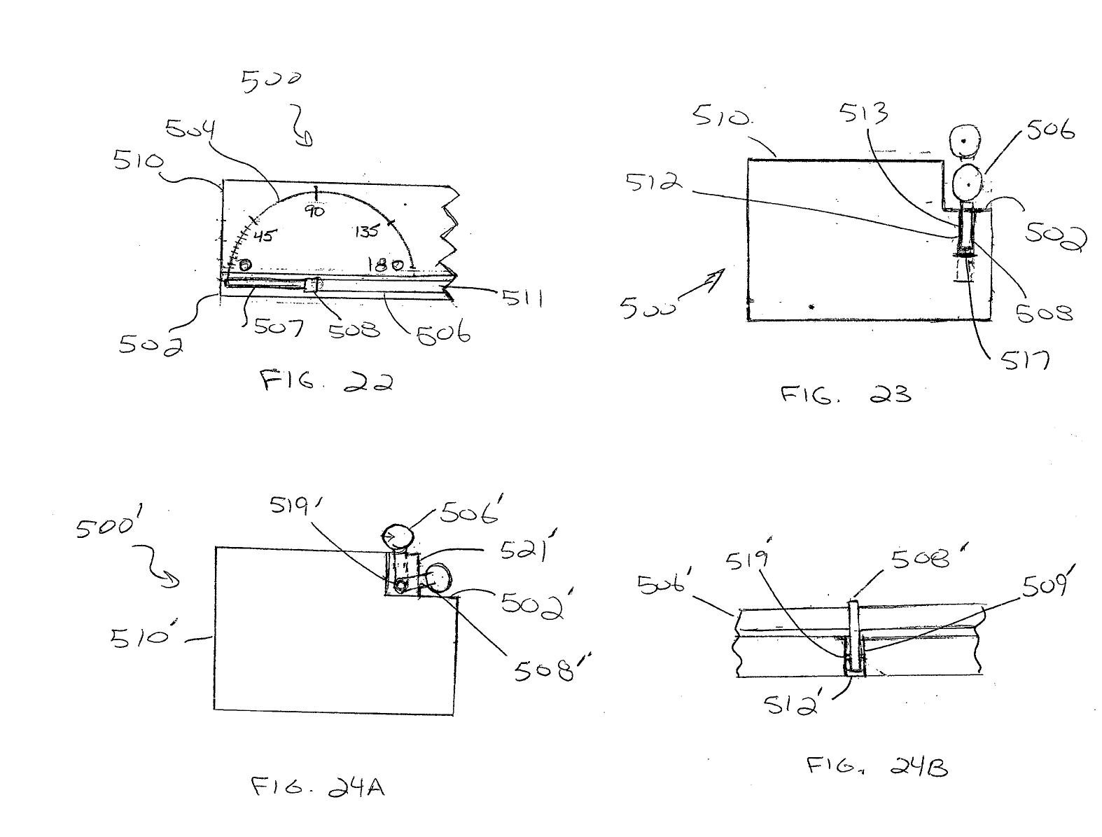

An alternative operating mechanism, including the axle 508′ and rod 506′, is shown in FIGS. 24A and 24B. In this version, the pocket arrangement is the same for the rod 506′ at the side of the instrument 500′. There is an anchoring slot 509′ that extends through a side wall 521′ of the pocket 502′. This allows the base of the axle 508′ to be fastened to a bolt or pin 519′ that is fixed to the elongate body member 510′. In turn, the axle 508′ can rotate thru a 90 degree angle and project either above the flat, top surface of the instrument 500′ and scale 504 (FIG. 22) or pivot down to lie within the pocket 502′. In this case, the connection of the rod 506′ to the axle 508′ allows rotation of the rod 506′ relative to the axle 508′. For example, the axle 508 can pass through an opening through the rod 506 and be secured thereto by a pin or other mechanical arrangement that allows the rod 506′ to rotate relative to the axle 508′.

Operation of the goniometer 501 involves pulling the rod 506 out of the pocket 502 with a finger tip. At this point, the rod 506 clears the top surface of the instrument 500 and is free to turn on the axle 508. In the alternative design, the rod 506′, at the point where the axle 508′ extends into the elongate body member 510′, is pivoted onto the top surface. At which point, the rod 506′ clears the top surface of the instrument and is free to turn on the axle 508′. The rod 506 rotates relative to the scale 504 on the top surface of the instrument, which provides an indication of the angular position of the rod 506. The scale 504 can include LED indicators as previously described.

During operation, the measuring end 511 of the rod 506 is placed on the surface to be measured and the marking end 507 lies along the scale 504. In this way, the marking end 507 of the rod 506 lies over the scale 504. The appropriate degree of angulation can be noted from the position of the marking end 507. When finished, the rod 506 can be rotated back into the pocket after turning it to lie parallel with the pocket 502.

Generally, the elongate body member 10 can be manufactured by injection molding or by modifying an extruded tube. For example, extrusion can be used to provide a uniform polymeric body, to which other components are attached, for example, the needles and mechanism. Insert molding can then be used to provide the desired geometry of openings in the elongate body member 10, or the openings can be created in the desired locations as a subsequent mechanical operation.

The elongate body member 10 and related components can be manufactured of polymeric materials, such as, for example, polyurethane, silicones, polyethylenes, nylons, polyesters and polyester elastomers, either with or without reinforcement. Steel, aluminum, brass, and stainless steel can also be used for the elongate body member 10 or various mechanical components. Other suitable materials will be apparent to those skilled in the art. Additionally, at least a portion of the elongate body member 10 can be transparent to facilitate viewing of a portion of the mechanism 20, for example, the indicator needle 50.

In one embodiment the dimensions of the elongate body member are about 12 cm to about 15 cm (5-6 inches) in length, about 1.5 cm to about 2 cm (0.6-0.8 inches) in width, and about 1.2 cm to about 1.5 cm (0.5-0.6 inches) in thickness. The various other components of the instrument will be sized as appropriate for the size and shape of the elongate body member and their particular function.

Having described certain embodiments of the invention, it will be apparent to those of ordinary skill in the art that other embodiments incorporating the concepts disclosed herein may be used without departing from the spirit and scope of the invention. The described embodiments are to be considered in all respects as only illustrative and not restrictive.

What is claimed is:

1. A medical instrument for evaluation of two-point discrimination, the instrument comprising:
an elongate body member;
a first needle comprising a distal tip, the first needle fixedly coupled to, and extending from a distal portion of the elongate body member;
a second needle comprising a distal tip, the second needle movably coupled to the elongate body member; and
a mechanism coupled to the elongate body member and the second needle, the mechanism configured to move the distal tip of the second needle relative to the distal tip of the first needle.

2. The instrument of claim 1, wherein the mechanism is disposed at least partially within the elongate body member.

3. The instrument of claim 1, further comprising a gauge configured to indicate a distance between the distal tip of the first needle and the distal tip of the second needle.

4. The instrument of claim 3, wherein the gauge is operatively coupled to the mechanism.

5. The instrument of claim 1, wherein a range of movement of the distal tip of the second needle relative to the distal tip of the first needle is from about 2 mm to about 12 mm.

6. The instrument of claim 1 further comprising a cover slidably coupled to the elongate body member, the cover slidable between a first position at least partially covering the first and second needles and a second position exposing the first and second needles.

7. The instrument of claim 1, wherein the mechanism comprises at least one of a worm drive, a rotary actuator, a linear actuator, a gear train, a linkage, a cam and follower, a bifurcated beam and splitter arrangement, and combinations thereof.

8. The instrument of claim 1, wherein the movement of the second needle relative to the first needle is by at least one of linear displacement and angular displacement.

9. The instrument of claim 1 further comprising an illumination source.

10. The instrument of claim 9, wherein the illumination source is a light emitting diode.

11. The instrument of claim 1, wherein the first needle and the second needle comprise an arcuate shape.

12. The instrument of claim 1, further comprising a pocket clip.

13. The instrument of claim 1, further comprising a scale disposed along a side of the elongate body member.

14. The instrument of claim 1, wherein the elongate body member comprises a power source.

15. The instrument of claim 14, wherein the power source is a battery.

16. The instrument of claim 1 further comprising a goniometer.

17. The instrument of claim 1, wherein the elongate body member comprises at least one of a molded plastic, a metal, and combinations thereof.

18. A medical instrument for evaluation of two-point discrimination, the instrument comprising:
an elongate body member;
a mechanism coupled to the elongate body member and at least partially disposed in a distal portion thereof;
a first needle coupled to the mechanism and extending from the distal portion of the elongate body member; and
a second needle coupled to the mechanism and extending from the distal portion of the elongate body member, wherein the mechanism is configured to provide movement to at least one of the first needle and the second needle to vary a distance between the first needle and the second needle.

19. The instrument of claim 18, wherein the mechanism is configured to provide substantially symmetrical movement of the first needle and the second needle relative to the elongate body member.

20. The instrument of claim 18, wherein the mechanism is configured to provide substantially concomitant movement of the first needle and the second needle.

21. The instrument of claim 18, wherein the mechanism comprises at least one of a worm drive, a rotary actuator, a linear actuator, a gear train, a linkage, a cam and follower, a bifurcated beam and splitter arrangement, and combinations thereof.

22. A medical instrument for evaluation of two-point discrimination, the instrument comprising:
an elongate body member;
a mechanism coupled to and at least partially disposed in the elongate body member;
a first needle comprising a distal tip, the first needle coupled to the mechanism and extending from a distal portion of the elongate body member;
a second needle comprising a distal tip, the second needle coupled to the mechanism and extending from the distal portion of the elongate body member, wherein the mechanism is configured to provide movement to at least one of the first needle and the second needle to vary a distance between the first needle and the second needle; and
a gauge disposed on the elongate body member and configured to indicate a distance between the distal tip of the first needle and the distal tip of the second needle.
